(19) United States
(12) Patent Application Publication
(10) Pub. No.: US 2012/0046249 A1
(43) Pub. Date: Feb. 23, 2012

(54) METHODS OF REDUCING THE RISK OF CARDIOVASCULAR DISEASE IN POSTMENOPAUSAL WOMEN

(75) Inventors: Lorne M. Golub, Smithtown, NY (US); Jeffrey B. Payne, Lincoln, NE (US)

(73) Assignee: The Research Foundation of State University of New York, Albany, NY (US)

(21) Appl. No.: 13/126,009

(22) PCT Filed: Nov. 4, 2009
(86) PCT No.: PCT/US09/63308

§ 371 Date: Nov. 10, 2011

Related U.S. Application Data
(60) Provisional application No. 61/111,171, filed on Nov. 4, 2008.
(60) Provisional application No. 61/115,315, filed on Nov. 17, 2008.

(51) Int. Cl. A61K 31/65; A61P 9/00

(52) U.S. Cl. 514/152

(57) ABSTRACT

The present invention features materials and methods for reducing the risk of cardiovascular disease in postmenopausal or perimenopausal women. More specifically, these methods can be used in women who generally have no apparent cardiovascular disease. We describe herein methods of administering non-antibacterial tetracycline or a sub-antimicrobial amount of antibacterial tetracyclines or tetracycline formulations and their use in reducing the risk that cardiovascular disease will develop in a subject (e.g., in a post- or perimenopausal woman)

### CROSS-REFERENCE TO RELATED APPLICATIONS

This application claims priority to U.S. Application Ser. No. 61/111,171, which was filed on Nov. 4, 2008 and U.S. Application Ser. No. 61/115,314, which was filed on Nov. 17, 2008. For the purpose of any U.S. patent that may issue based on the present application, U.S. Application Ser. No. 61/111,171 and U.S. Application Ser. No. 61/115,314 are hereby incorporated by reference herein in their entirety.

### FEDERALLY SPONSORED RESEARCH OR DEVELOPMENT

This invention was made with government support under grant number R01DE012872 awarded by the National Institute of Dental and Craniofacial Research at the National Institutes of Health. The government has certain rights in the invention

### TECHNICAL FIELD

This invention relates to methods and compositions useful for reducing the risk of cardiovascular disease and more particularly to methods of treating post- or perimenopausal women.

### BACKGROUND

Cardiovascular diseases are a group of disorders of the heart and blood vessels and include, for example, coronary heart disease (i.e., disease of the blood vessels supplying the heart muscle); cerebrovascular disease (i.e., disease of the blood vessels supplying the brain); peripheral arterial disease (i.e., disease of blood vessels supplying the arms and legs); and deep vein thrombosis and pulmonary embolism (i.e., blood clots in the leg veins, which can dislodge and move to the heart and lungs). Cardiovascular diseases claim more than 17.1 million lives a year worldwide. In the United States, cardiovascular diseases are responsible for 40 percent of all the deaths, more than all forms of cancer combined. Many forms of heart disease can be prevented or treated with healthy lifestyle choices, diet and exercise. There is a continuing need for therapeutic strategies that reduce the development of cardiovascular disease.

### SUMMARY OF THE INVENTION

The present invention features methods of reducing the risk of cardiovascular disease in a subject (e.g., a post- or perimenopausal woman) who has no apparent cardiovascular disease. The methods can be carried out by administering, to the subject, an effective amount of a tetracycline formulation, which may include either a non-antibacterial tetracycline or a sub-antibacterial or sub-antimicrobial amount or concentration of an antibacterial tetracycline. As described further below, the formulations can include more than one type of tetracycline compound and/or more than one type of a pharmaceutically acceptable salt thereof. Where a non-antibacterial tetracycline is used, it may be a chemically modified tetracycline compound or a pharmaceutically acceptable salt thereof. For ease of reading, we will not repeat the phrase “or a pharmaceutically acceptable salt thereof” on every occasion. It is to be understood that where a tetracycline compound can be used, whether chemically modified or not, a pharmaceutically acceptable salt of the compound may also be used.

Where one administers an antibacterial tetracycline in an amount that is too low for the tetracycline to exert an antibacterial effect (i.e., a sub-antibacterial or sub-antimicrobial amount), one can describe the amount (whether expressed in terms of an absolute amount, dosage or concentration) relative to an amount of the tetracycline that does produce an antibacterial effect. For example, the amount, dose, or concentration of a sub-antimicrobial tetracycline formulation can be up to about 80% (e.g., about 10-80% (e.g., about 50%, 60%, or 70%)) of the amount, dose or concentration of a corresponding antibacterial tetracycline formulation.

The antibacterial tetracycline compound can conform to Formula (I):

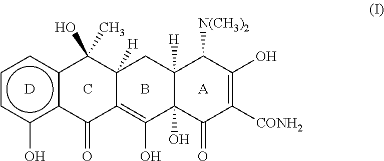

For example, the antibacterial tetracycline compound can be an oxytetracycline or chlorotetracycline or a pharmaceutically acceptable salt thereof. The antibacterial tetracycline can also be 7-dimethylaminotetracycline (minocycline) or 6α-deoxy-5-hydroxytetracycline (doxycycline) or pharmaceutically acceptable salts thereof.

As noted, the present methods can also be carried out with a chemically modified tetracycline that has little or no antibacterial activity. For example, the chemically modified tetracycline compound can differ from Formula (I) by a change to the basic ring system or replacement of one or more of the substituents at positions 4, 10, 11, 12 or 12a according to Formula II:

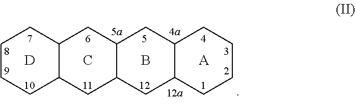

In specific embodiments, the chemically modified tetracycline used according to the methods described herein can be: 4-dedimethylaminotetracycline (CMT-1); 6-demethyl-6-deoxy-4-de(dimethyl-amino)tetracycline (CMT-3); 7-chloro-4-de(dimethylamino)tetracycline (CMT-4); 4-hydroxy-4-de(dimethylamino)-tetracycline (CMT-6); 4-de(dimethylamino)-12a-deoxytetracycline (CMT-7); 6-deoxy-5α-hydroxy-4-de(dimethylamino)tetracycline (CMT-8); 4-dedimethylamino-12α-deoxyanhydrotetracycline (CMT-9); 7-dimethyl-amino-6-demethyl-6-deoxy-4-de(dimethylamino)tetracycline (CMT-10); 4-dedimethyl-amino-5-oxytetracycline; 5α,6-anhydro-4-hydroxy-4-de(dimethylamino)tetracycline; 4-de(dimethylamino)-11-hydroxy-12α-deoxytetracycline; 12α-deoxy-4-deoxy-4-de(dimethylamino)tetracycline; 12α,4α-anhydro-4-de(dimethylamino)tetracycline; 6-α-benzylthiomethylenetetracycline; 6-fluoro-6-demethyltetracycline; 11α-chlorotetra-cycline; tetracyclinonitrile (CMT-2); 9-amino-6-demethyl-6-deoxy-4-de-dimethylaminotetracycline (CMT-308 (i.e., 9-amino CMT-3)); or tetracycline pyrazole (CMT-5).

The pharmaceutically acceptable salt can be an acid addition salt, such as an acid addition salt formed with a mineral acid (e.g., hydrochloric acid, hydriodic acid, hydrobromic acid, phosphoric acid, metaphosphoric acid, nitric acid, or sulfuric acid). The acid addition salt can also be formed with an organic acid (e.g., tartaric acid, acetic acid, citric acid, malic acid, benzoic acid, glycolic acid, gluconic acid, gulonic acid, succinic acid, or arylsulfonic acid).

Whether or not the subject (e.g., a peri- or postmenopausal woman) has apparent cardiovascular disease can be determined by performing one or more diagnostic tests. For example, one can assess a sample obtained from the patient for a marker of cardiovascular disease. The marker can indicate systemic inflammation, and such markers include C reactive protein (CRP), TNFa receptors, interleukins (e.g., IL-6) and other pro-inflammatory cytokines. The marker can also be a gene whose expression is correlated with a marker of systemic inflammation. For example, expression of the marker CRP is correlated with genes encoding hepatocyte nuclear factor-1α (HNF1A), genes encoding a leptin receptor, and genes encoding apolipoprotein E. The marker can also be a matrix metalloproteinase such as MMP-2 or MMP-9. Increased troponin I indicates damage or necrosis of cardiac myocytes. Expression of any of these genes or the encoded proteins can be assessed, either alone or in combination, and the assessment step can be a step in any of the present methods.

There is no apparent cardiovascular disease when the results of the diagnostic tests (e.g., the levels of expression of the genes indicating systemic inflammation) are within normal limits. As often happens in the practice of medicine, a physician may consider the results of more than one type of diagnostic test and come to a decision regarding treatment based on the totality of the circumstances and in consultation with his or her patient. Thus, the present methods can be used to reduce the risk of cardiovascular disease where a diagnostic test (or tests) indicate that there is no apparent cardiovascular disease and that the subject's risk is not elevated (e.g., not more than one would expect for a given subject (e.g., not more than one would expect for a peri- or postmenopausal woman)).

In other diagnostics, one can obtain medical images and/or perform a physical examination or test. For example, cardiovascular disease can be assessed by obtaining an X-ray image (e.g., of the patient's chest, a chamber of the heart or the lumen of a blood vessel). The physical examination can include assessing blood pressure, body mass index, family history, or the electrical activity of the heart. In addition, biochemical measures, such as cholesterol levels, can be assessed as indicators of risk for future cardiac events (including acute myocardial infarction). The heart can also be assessed using physical tests such as a stress test. As noted, the present methods can be implemented in a subject who scores well in these tests (e.g., blood pressure and body mass index within recommended guidelines) and can serve to increase the likelihood that the subject will remain free of cardiovascular disease or that any disease that may develop will be less severe than it otherwise would have been. Thus, the present methods can be characterized as methods of reducing the risk of cardiovascular disease or as methods of increasing the likelihood that a subject will remain free or substantially free of cardiovascular disease.

In certain subjects, the levels of expression of genes associated with systemic inflammation may be normal (e.g., there may be little or no elevation in CRP expression (e.g., in a postmenopausal woman)), but a different indicator of cardiovascular disease may be present. Those subjects are amenable to treatment; the present invention encompasses methods of reducing the risk that cardiovascular disease will develop in a subject (e.g., a postmenopausal or perimenopausal woman) who has little or no elevation in the expression of a gene associated with systemic inflammation (e.g., CRP). The subject may nevertheless have high blood pressure or an undesirable cholesterol profile. In such cases, the tetracycline formulations described herein can be administered together with an anti-hypertensive agent or cholesterol-lowering drug.

More specifically, in the present methods, where indicated, the tetracycline formulation can be administered in combination with other agents such as an anti-hypertensive agent (e.g., a diuretic, an adrenergic receptor agonist or antagonist, a calcium channel blocker, an ACE inhibitor, an angiotensin II receptor antagonist, an aldosterone antagonist, a vasodilator, or a centrally acting adrenergic drug) or an HMG-CoA reductase inhibitor (e.g., atorvastatin, rosuvastatin, or simvastatin).

Any of the methods described herein for administering a tetracycline formulation to a subject can be presented in the form of a “use” claim. Accordingly, the invention features a tetracycline formulation for use in reducing the risk of cardiovascular disease in a subject (e.g., a post- or perimenopausal woman). Any of the tetracycline formulations described herein, including non-antibacterial tetracyclines and sub-antibacterial amounts of antibacterial tetracyclines can be used in the patients amenable to treatment (as described above and further below).

### DETAILED DESCRIPTION OF THE INVENTION

The present invention is based, in part, on the inventors' discovery that administering a non-antimicrobial tetracycline formulation could decrease the levels of CRP and MMP-9 in postmenopausal women with systemic osteopenia and periodontitis but with no apparent heart disease. As these serum inflammatory biomarkers are associated with risk of cardiovascular disease, the present methods are designed to reduce the risk of cardiovascular disease, particularly in the population of post- or perimenopausal women; the latter category is believed to be vulnerable to cardiovascular disease due to estrogen deficiency.

Tetracycline formulations: Tetracycline is a member of a class of antibiotic compounds variously referred to as the tetracyclines, tetracycline compounds, tetracycline derivatives, and the like. Tetracycline, as well as the terramycin and aureomycin derivatives, exist in nature, and are well known antibiotics. Naturally occurring tetracyclines can be modified without losing their antibiotic properties, although certain elements must be retained. Modifications that may and may not be made to the basic tetracycline structure have been reviewed by Mitscher (The Chemistry of Tetracyclines, Chapter 6, Marcel Dekker, New York (1978)). According to Mitscher, the substituents at positions 5-9 of the tetracycline ring system can be modified without a complete loss of antibiotic properties.

The present methods for reducing the risk of cardiovascular disease are carried out by administering an effective amount of a tetracycline formulation. As noted, the formulation can include an antibacterial tetracycline compound (which can be administered at a sub-antimicrobial dose) or a tetracycline compound that has been chemically modified to reduce its bacteriostatic activity. Whether modified or not, the tetracycline can be administered as a pharmaceutically acceptable salt, and any of the compounds can be combined with a pharmaceutical carrier.

Useful antibacterial tetracycline compounds include (but are not limited to) doxycycline, minocycline, tetracycline, oxytetracycline, chlortetracycline, demeclocycline, lymecycline and their pharmaceutically acceptable salts. Doxycycline can be administered in the form of its hyclate salt or as a hydrate (e.g., a monohydrate).

Useful non-antibacterial tetracycline compounds are structurally related to the antibacterial tetracyclines but have had their antibiotic activity substantially or completely eliminated by chemical modification. For example, changes to the basic ring system or replacement of the substituents at positions 4 and 10-12a, as shown in the following formula (Formula II) generally lead to synthetic tetracyclines with substantially less or effectively no antibacterial activity:

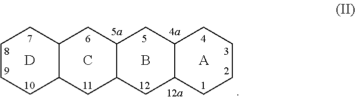

Non-antibacterial tetracycline compounds may be capable of exerting an antibacterial effect when used in an amount much higher than the amount at which a naturally-occurring tetracycline is useful as an antibiotic. For example, a non-antibacterial tetracycline compound may have activity comparable to that of tetracycline compounds when the concentration of the non-antibacterial compound is at least or about five times higher than that of the antibacterial compound (e.g., at least or about five, ten, or 25 times higher than the amount of doxycycline or minocycline).

Examples of chemically modified non-antibacterial tetracyclines include compounds lacking the dimethylamino group at position 4 of the tetracycline ring structure. For example, the non-antibacterial tetracycline can be: 4-dedimethylamino-tetracycline (CMT-1); 6-demethyl-6-deoxy-4-de(dimethylamino)tetracycline (CMT-3); 7-chloro-4-de(dimethylamino)tetracycline (CMT-4); 4-hydroxy-4-de(dimethylamino)-tetracycline (CMT-6); 4-de(dimethylamino)-12α-deoxytetracycline (CMT-7); 6-deoxy-5α-hydroxy-4-de(dimethylamino)tetracycline (CMT-8); 4-dedimethylamino-12α-deoxyanhydrotetracycline (CMT-9); 7-dimethylamino-6-demethyl-6-deoxy-4-de(dimethylamino)tetracycline (CMT-10); 4-dedimethylamino-5-oxytetracycline; 5α.,6-anhydro-4-hydroxy-4-de(dimethylamino)tetracycline; 4-de(dimethylamino)-11-hydroxy-12α-deoxytetracycline; 12α-deoxy-4-deoxy-4-de(dimethylamino)tetracycline; 9-amino-6-dimethyl-6-deoxy-4-de-dimethylaminotetracycline (CMT-308 (i.e., 9-amino CMT-3); and 12α,4α-anhydro-4-de(dimethylamino)tetracycline. Additional examples of tetracyclines modified for reduced antibacterial activity include 6-α-benzylthiomethylenetetracycline, the mono-N-alkylated amide of tetracycline, 6-fluoro-6-demethyltetracycline, 11α-chlorotetracycline, tetracyclinonitrile (CMT-2), and tetracycline pyrazole (CMT-5).

Further examples of generic and specific tetracycline compounds that are suitable for use in the methods of the invention are disclosed in international PCT application WO 01/87823, which is hereby incorporated by reference in its entirety.

Derivatives of non-antibacterial tetracyclines can also be used. These include derivatives of the compounds listed above (e.g., compounds in which a substituent is added to the 7, 8, or 9 position of the tetracycline ring nucleus). Examples of substituents include halo (e.g., F, Cl, Br, and I); nitro; hydroxy, alkyl carbonyl; alkyl carbonyloxy; alkyl amido; amino; alkyl amino; dialkyl amino; phenyl; and carboxylate. Alkyl groups can include 1-16 carbons (e.g., C₁-C₄) and can be straight chain or branched alkyl groups (e.g., methyl, ethyl, or isopropyl groups).

For example, some useful derivatives of CMT-3 include: 7-bromo-6-demethyl-6-deoxy-4-dedimethylaminotetracycline (CMT-301); 7-nitro-6-demethyl-6-deoxy-4-dedimethyl-aminotetracycline (CMT-302); 9-nitro-6-demethyl-6-deoxy-4-dedimethylamino-tetracycline (CMT-303); 7-acetamido-6-demethyl-6-deoxy-4-dedimethylamino-tetracycline (CMT-304); 9-acetamido-6-demethyl-6-deoxy-4-dedimethylamino-tetracycline (CMT-305); 9-dimethylamino-6-demethyl-6-deoxy-4-dedimethylamino-tetracycline (CMT-306); 7-amino-6-demethyl-6-deoxy-4-dedimethylaminotetracycline (CMT-307); 9-amino-6-demethyl-6-deoxy-4-dedimethylaminotetracycline (CMT-308); 9-dimethylaminoacetamido-6-demethyl-6-deoxy-4-dedimethylaminotetracycline (CMT-309); 7-dimethylamino-6-demethyl-6-deoxy-4-dedimethylaminotetracycline (CMT-310); 9-palmitamide-6-demethyl-6-deoxy-4-dedimethylaminotetracycline (CMT-311); 2-CONHCH₂-pyrrolidin-1-yl-6-demethyl-6-deoxy-4-dedimethylamino-tetracycline (CMT-312); 2-CONHCH₂-piperidin-1-yl-6-demethyl-6-deoxy-4-dedimethylaminotetracycline (CMT-313); 2-CONHCH₂-morpholin-1-yl-6-demethyl-6-deoxy-4-dedimethylaminotetracycline (CMT-314); and 2-CONHCH₂-piperazin-1-yl-6-demethyl-6-deoxy-4-dedimethylaminotetracycline (CMT-315). CMT is an abbreviation for chemically modified tetracycline.

Some useful derivatives of CMT-8 include: 9-acetamido-4-dedimethylamino-doxycycline (CMT-801); 9-dimethylaminoacetamido-4-dedimethylaminodoxycycline (CMT-802); 9-palmitamide-4-dedimethylaminodoxycycline (CMT-803); 9-nitro-4-dedimethylaminodoxycycline (CMT-804); 9-amino-4-dedimethylaminodoxycycline (CMT-805); 9-dimethylamino-4-dedimethylaminodoxycycline (CMT-806); 2-CONHCH₂-pyrrolidin-1-yl-4-dedimethylaminodoxycycline (CMT-807); 2-CONHCH₂-piperidin-1-yl-4-dedimethylaminodoxycycline (CMT-808); and 2-CONHCH₂-piperazin-1-yl-4-dedimethylaminodoxycycline (CMT-809).

Some useful derivatives of CMT-10 include: 7-trimethylammonium-4-dedimethylaminosancycline (CMT-1001) and 9-nitro-4-dedimethylaminominocycline (CMT-1002).

Pharmaceutically acceptable salts of the compounds described herein are salts that do not substantially contribute to the toxicity of the compound. Such salts can be formed by well known procedures and include acid addition salts of basic tetracycline compounds. The acid can be a mineral acid (e.g., hydrochloric, hydriodic, hydrobromic, phosphoric, metaphosphoric, nitric or sulfuric acid) or an organic acid (e.g., tartaric, acetic, citric, malic, benzoic, glycollic, gluconic, gulonic, succinic, or arylsulfonic acid).

Effective amounts: The amount of the tetracycline formulation administered in the present methods can be described as an “effective” amount (i.e., an amount effective in reducing the risk of cardiovascular disease). As the methods are applied to patients who may be considered at risk for cardiovascular disease but who do not as yet have any apparent cardiovascular disease, and as CRP is considered a risk factor for cardiovascular disease, the “effective amount” of the formulation can be an amount that lowers a patient's CRP levels or favorably impacts another indicator of cardiovascular disease (e.g., increases the blood levels of HDL (“good”) cholesterol.

The actual amounts of the tetracycline formulation administered to a particular individual can vary according to various factors that are routinely considered in the art, such as the particular compound(s) formulated, the mode of administration, and the individual being treated. In determining dosage, one may consider the minimal amount of tetracycline in a given formulation that is capable of decreasing a minimally elevated (>3 μg/ml indicates significant risk) level of CRP and the highest effective amount that does not cause undesirable or intolerable side effects.

The tetracycline formulations can include an amount of tetracycline that is effective in decreasing “low risk” levels of CRP and/or in reducing the risk of developing cardiovascular disease but that has substantially no antibacterial activity (i.e., does not prevent or significantly prevent the growth of bacteria). Administering sub-antimicrobial doses of tetracycline can reduce the risk of antibiotic resistance. Sub-antibacterial amounts of antibacterial tetracycline compounds may be described relative to bacteriostatic amounts. For example, a formulation may have up to or about 10% (e.g., 10%, 10-80%, 20-60%, 20-60%, 25%, 30%, 35%, 40%, 50%, 55%, 60%, 65%, 70% or 80%) of the tetracycline of a corresponding antibacterial formulation.

More specifically, the present methods encompass administration of an antibacterial tetracycline compound at doses of: 100 mg/day (e.g., 100 mg/day of doxycycline or minocycline), 250 mg four times per day (e.g., 250 mg of tetracycline four times a day), 1000 mg/day (e.g., 1000 mg/day of oxytetracycline), or 600 mg/day (e.g., 600 mg/day of demeclocycline or lymecycline). An effective amount of a non-antibacterial tetracycline compound can be from about 1.0 mg/day to about 2000 mg/day.

Chemical Synthesis: Chemically modified tetracycline compounds can be synthesized by methods used routinely in the chemical arts. For guidance, one of ordinary skill in the art can consult Mitscher, L. A. (The Chemistry of the Tetracycline Antibiotics, Chapter 6, Marcel Dekker, New York (1978)) and U.S. Pat. Nos. 4,704,383 and 5,532,227. After synthesis, the compounds can be conveniently purified by standard methods known in the art. Suitable purification methods include crystallization from a suitable solvent or partition-column chromatography.

Assessing cardiovascular disease and patients amenable to treatment: The present methods can be applied to any subject (or patient) who is at risk of developing cardiovascular disease (e.g., patients for whom physicians would recommend preventative treatment out of concern for their near-term health) but who has little or no apparent cardiovascular disease at the time treatment commences. We use the term “apparent” as it is normally used to refer to a condition that is clearly revealed. We may also refer to subjects in which there is no overt (open) or frank (clearly manifest) cardiovascular disease. The patient may have a heightened risk for cardiovascular disease, yet show no evidence of such disease (e.g., the patient's cholesterol and CRP levels may be within normal limits or near-normal). More specifically, the patient can be a post- or perimenopausal woman, an older man (e.g., a man over about 55 years of age), or a patient from a family with a high incidence of cardiovascular disease. In some embodiments, the man or woman being treated can be about or between 45 and 75 years old. While the present methods would benefit human patients, the invention is not so limited. Any mammal, including a domesticated farm animal or pet, can be treated.

The cardiovascular disease can be any disease of the heart, as well as disorders of the blood vessels. An example of a patient who is at risk for cardiovascular disease is a patient who has an increased risk of myocardial infarction, cerebrovascular accident, or hypercholesterolemia.

Whether or not a patient has apparent cardiovascular disease can be dtermined by performing one or more diagnostic tests. For example, one can assess a sample obtained from the patient for a marker of cardiovascular disease. The marker can indicate systemic inflammation, and such markers include C reactive protein (CRP), TNFα receptors, and interleukins (e.g., IL-6) and other pro-inflammatory cytokines. The marker can also be a protein detected in serum samples that indicates cardiac damage (e.g., troponin). The marker can also be a gene whose expression is correlated with a marker of systemic inflammation. For example, expression of the marker CRP is correlated with genes encoding hepatocyte nuclear factor-1α (HNF1A), genes encoding a leptin receptor, and genes encoding apolipoprotein E. Any of these genes can be assessed, either alone or in combination.

In other diagnostics, one can obtain medical images and/or perform a physical examination or test. For example, cardiovascular disease can be assessed by obtaining an X-ray image (e.g, of the patient's chest, a chamber of the heart or the lumen of a blood vessel). The physical examination can include assessing blood pressure, cholesterol levels, body mass index, family history, or the electrical activity of the heart. The heart can also be assessed using physical tests such as a stress test.

Tetracyclines have a number of uses other than those based on their antibacterial properties. For example, tetracyclines inhibit the activity of collagen destructive enzymes produced by mammalian cells and tissues. These enzymes include the matrix metalloproteinases (MMPs), including collagenases (MMP-1, MMP-8 and MMP-13), gelatinases (MMP-2 and MMP-9), and others (e.g., MMP-12 and MMP-14). See Golub et al. (J. Periodont. Res. 20:12-23, 1985); Golub et al. (Crit. Revs. Oral Biol. Med. 2:297-322, 1991); and U.S. Pat. Nos. 4,666,897; 4,704,383; 4,935,411; and 4,9354,412. Tetracyclines have also been shown to inhibit wasting and protein degradation in mammalian skeletal muscle (U.S. Pat. No. 5,045,538); to inhibit inducible NO synthase (U.S. Pat. Nos. 6,043,231 and 5,523,297); to inhibit phospholipase A₂ (U.S. Pat. Nos. 5,789,395 and 5,919,775); to enhance anti-inflammatory IL-10 production in mammalian cells (U.S. Pat. No. 6,015,804); and to reduce elevated serum plasma LDL-cholesterol levels and CRP levels (U.S. Pat. No. 6,841,547; but see Korpela et al., J. Gastroenterol. 19:401-404, 1984; Samuel et al., Circ. Res. 33:393-402, 1973; and Berchev et al.).

The activities described above have led to the use of tetracyclines (or their suggested use) in treating a number of diseases or conditions, and the methods of the present invention can exclude the treatment of patients that have a disease or condition that was previously known to be treatable with a modified tetracycline that has little or no antibacterial activity or a sub-antimicrobial dose of an antibacterial tetracycline. Accordingly, the patient population may exclude patients who have acne, rosacea, an aneurysm (e.g., an abdominal aortic aneurysm), ulceration of the cornea, periodontal disease, diabetes, scleroderma, progeria, lung disease, cancer, graft versus host disease, a disease of depressed bone marrow function, thrombocytopenia, prosthetic joint loosening, a spondyloarthropathy, osteoporosis, Paget's disease, an autoimmune disease, systemic lupus erythematosus, an acute or chronic inflammatory condition, a renal disease, a connective tissue disease, or a neurological or neurodegenerative condition. Other patients who can be excluded from treatment with the present methods may have a condition featuring telangiectasias (e.g., advanced age, excessive sun exposure, alcohol abuse, scleroderma, hereditary hemorrhagic telangiectasia (Olser-Rendu syndrome), ataxia-telangiectasia, spider angioma, cutis marmorata telangiectasia congenita, Bloom syndrome, Klippel-Trenaunay-Weber syndrome, Sturge-Weber disease, xeroderma pigmentosa or nevus flammeus). Accordingly, the present methods include reducing the risk of cardiovascular disease in a patient by administering to the patient an effective amount of a non-antibacterial tetracycline compound or a pharmaceutically acceptable salt thereof, and the patient may have no apparent cardiovascular disease and no condition that is being treated with, or is known to be treatable with, a non-antibacterial tetracycline or a sub-antimicrobial dose of an antibacterial tetracycline (including any one or more of the conditions just listed).

Modes of Administration: The tetracycline formulation may be administered alone or as an adjunct with other conventional drugs for lowering the risk of cardiovascular disease or for otherwise maintaining the health of a patient (e.g., a post- or perimenopausal woman).

The tetracycline formulations may be administered by any method known in the art, including by oral or parenteral routes. Given that the present formulations can be self-administered, oral or enteral administration is a preferred route of delivery, and the tetracycline formulations can be formulated as liquids or solids that can be swallowed. Some examples of formulations suitable for oral administration are tablets, capsules (e.g., gelatin capsules), pills, troches, elixirs, suspensions, syrups, and wafers.

Tetracycline formulations intended for parenteral administration include, for example, intravenous, intramuscular, and subcutaneous injections. Other routes of administration include topical, intrabronchial, and intranasal administration. Intrabronchial administration can be facilitated by an inhaler spray, and intranasal administration can be accomplished by a nebulizer or liquid mist. The formulations may also result in sustained release, thereby achieving a certain level of the tetracycline over a particular period of time.

The tetracycline formulations can include not only one or more tetracycline compounds, but also a suitable pharmaceutical carrier. The term “carrier,” as used herein, is synonymous with a “vehicle” or an “excipient” unless otherwise noted. Exemplary carriers include starch, milk, sugar, certain types of clay, gelatin, stearic acid or salts thereof, magnesium or calcium stearate, talc, vegetable fats or oils, gums and glycols.

The tetracycline formulations may also include one or more of the following: a stabilizer, a surfactant (e.g., a nonionic surfactant such as polysorbate), a salt or a buffering agent. The stabilizer may be, for example, an amino acid (e.g., glycine), an oligosaccharide (e.g., sucrose, tetralose, lactose or a dextran), a sugar alcohol (e.g., mannitol), or a combination thereof The stabilizer or combination of stabilizers may constitute from about 0.1% to about 10% (w/w) of the tetracycline formulation. Other examples of suitable surfactants include Tween 20, Tween 80, a polyethylene glycol, and a polyoxyethylene polyoxypropylene glycol (e.g., Pluronic F-68). The stabilizer can constitute from about 0.001% (w/v) to about 10% (w/v) of the fog ululation.

The salt or buffering agent may be essentially any salt or buffering agent, including, for example, sodium chloride or sodium/potassium phosphate, respectively (but not cations such as calcium). Preferably, the buffering agent maintains the pH of the tetracycline formulation in the range of about 5.5 to about 7.5. The salt and/or the buffering agent can also serve to maintain the osmolality at a level suitable for administration to a patient and can be present at roughly isotonic concentrations (e.g., concentrations of about 150 mM to about 300 mM).

The tetracycline formulations may additionally contain one or more conventional additives. Some examples of such additives include a solubilizer (e.g., glycerol), an antioxidant (e.g., benzalkonium chloride (a mixture of quaternary ammonium compounds, known as “quart”)), benzyl alcohol, chloretone or chlorobutanol, an anaesthetic agent (e.g., a morphine derivative), and an isotonic agent. As a further precaution against oxidation or other spoilage, the tetracycline fommlations may be stored under nitrogen gas in vials sealed with impermeable stoppers.

Combination therapies: The present methods can include treating the patient with a second agent (i.e., a non-tetracycline) in order to further reduce the risk of developing cardiovascular disease. For example, the methods can include treatment as described above and treatment with an anti-hypertensive agent or an HMG-CoA reductase inhibitor. The anti-hypertensive agent can be a diuretic, an adrenergic receptor agonist or antagonist, a calcium channel blocker, an ACE inhibitor, an angiotensin II receptor antagonist, an aldosterone antagonist, a vasodilator, or a centrally acting adrenergic drug. The HMG-CoA reductase inhibitor can be a “statin” such as atorvastatin, rosuvastatin, or simvastatin. Statins generally alter the metabolism of various constituents within the cholesterol metabolic pathway and typically reduce serum/plasma LDL-cholesterol levels and CRP levels. Statins are associated with numerous side effects, including elevation of plasma triglycerides, increased liver aminotransferase activity, abdominal discomfort, nausea, vomiting, diarrhea, malaise, QT interval prolongation, and decreased high-density lipoprotein levels. These side effects may be reduced where a patient is treated with a tetracycline compound as described herein because the use of a sub-antimicrobial tetracycline compound or formulation could decrease the dose of the statin needed to reduce CRP (and other markers) to low therapeutically desirable levels.

### EXAMPLES

### Example 1

### Serum Gelatinases/Type IV Collagenases in Postmenopausal, Osteopenic Women with Periodontitis: Sub-antimicrobial-dose-doxycycline (SDD)

The objective of this study was to determine the effect of a long-term regimen of SDD on systemic levels of matrix metalloproteinases (MMP-2 and MMP-9) in postmenopausal (PM) women who exhibited both local (periodontitis) and mild systemic bone loss (osteopenia). Elevated circulating MMP 9 has been associated with increased risk for fatal cardiac events and PM are considered a population vulnerable, due to decreased estrogen levels, to cardiovascular disease.

Details concerning subject inclusion and exclusion criteria have been published previously (Payne et al., J. Clin. Periodontol. 34:776-787, 2007). Briefly, subjects were 45-70 years of age at telephone screening, postmenopausal, osteopenic at the lumbar spine or femoral neck, and not receiving hormone replacement therapy (HRT). The subjects had a history of generalized moderate to advanced periodontitis and were undergoing periodontal maintenance. The subjects also had to be in good general health without co-morbidities that could have interfered with adherence to the study protocol, planned follow-up or endpoint measurement. Subjects were excluded if they had an allergy or hypersensitivity to tetracyclines, had diseases or regular drug therapy that would affect the inflammatory or immune response, had active periodontal therapy within the past year, had diabetes, or had osteoporosis at either the lumbar spine or femoral neck.

113 PM women who completed the randomized clinical trial (RCT) consented at the final (two-year) study visit for the analysis of stored serum samples. 51 subjects received SDD (20 mg doxycycline) and 62 received placebo tablets b.i.d. for 2 years. Serum samples were collected at the baseline, 1-year and 2-year appointments and frozen at −80° C. until analyzed. MMPs were measured by gelatin zymography using denatured type I collagen as a substrate and purified MMP-2 (72 kDa) and MMP-9 (92 kDa) as standards. Gelatinolytic bands were scanned densitometrically. Statistical analyses were performed using Generalized Estimating Equations. Primary analyses were intent-to-treat (ITT). Per-protocol analyses were also performed. All results are presented as difference in means (SDD minus placebo).

Based on ITT and per-protocol analyses, the 2-year regimen of SDD reduced serum MMP-9 levels by 28.4 and 29.4 scanning units, respectively. Both reductions were highly statistically significant (p>0.0001). The higher molecular weight forms (MMP-9 homodimer) of this gelatinase were not significantly affected (p=0.5). Changes in serum levels of MMP-2 showed a similar trend to 92 kDa gelatinase; however only the 2-year MMP-2 values were significantly reduced by an average of 16.5 units (p=0.03; per-protocol analysis).

In conclusion, a 2-year regimen of SDD in PM women, exhibiting mild systemic bone loss and local bone loss, significantly reduced systemic MMP-9 levels and, based on per-protocol analyses, MMP-2. Based on previous large studies, reduction in circulating MMP-9 could diminish the risk for serious cardiac events (i.e., fatal heart attacks) in this vulnerable PM population.

### Example 2

### Sub-antimicrobial-dose-doxycycline Effects on Serum Inflammatory Biomarkers in Postmenopausal, Osteopenic Women with Periodontitis

The objective of this study was to determine whether sub-antimicrobial-dose-doxycycline (SDD) can reduce serum inflammatory biomarkers associated with cardiovascular disease (CVD) risk (primary outcome: CRP) in a two-year, randomized controlled clinical trial (RCT) in postmenopausal (PM) women with systemic osteopenia and periodontitis.

113 women who completed the RCT (SDD group: n=51; placebo group: n=62) consented at the final (two-year) study visit for their baseline, one-year and two-year serum samples to be analyzed for serum inflammatory biomarkers. Analyses by ELISA included CRP (high-sensitivity ELISA), IL-6, myeloperoxidase (MPO), IL-1β and TNF-α. Serum lipids (total cholesterol, HDL cholesterol, LDL cholesterol, VLDL cholesterol, and triglycerides) were analyzed by a commercial laboratory. Statistical analyses were performed using Generalized Estimating Equations; primary analyses were intent-to-treat (ITT). Pre-specified subgroup analyses also were performed. All results are presented as ratios of median values (SDD versus placebo) unless otherwise indicated.

By ITT, median CRP levels were reduced by 18% for SDD subjects compared to placebo, which was statistically significant (0.82, 95% Cl: 0.70 to 0.97; p=0.02). There was no significant difference between groups with respect to IL-6, MPO, and serum lipids based on ITT. IL-1β was not detectable in any serum samples, and TNF-α levels were below assay detection limits in 68% of serum samples. In women more than 5 years postmenopausal, SDD was significantly associated with an increase in HDL cholesterol over time (difference in means [mg/dl]: 5.99; 95% Cl: 1.17 to 10.81, p=0.01). In the same subgroup, SDD treatment was marginally associated with a decrease in VLDL cholesterol (0.87, 95% Cl: 0.76 to 1.00; p=0.06) and triglycerides (0.87, 95% Cl: 0.76 to 1.01; p=0.06).

In conclusion, in a two-year RCT in PM women, SDD treatment, relative to placebo, resulted in improvement in several serum inflammatory biomarkers associated with CVD risk.

### Example 3

### Sub-Antimicrobial-Dose-Doxycycline Reduces the Media MMP-8/TIMP-1 Ratio

Based on further analysis of the study described above, we found that, in women within five years of menopause, SDD reduced the median MMP-8/TIMP-1 ratio by 49% at two years (ratio of medians [SDD relative to placebo]: 0.51; 95% Cl: 0.31 to 0.82, p=0.006. MMP-8 is a tissue destructive enzyme, and TIMP-1 is its natural inhibitor. The reduction we observed is highly statistically significant and indicates a reduction in the potential for tissue destruction.

1. A method of reducing the risk of cardiovascular disease in a post- or perimenopausal woman who has no apparent cardiovascular disease, the method comprising administering to the woman an effective amount of a tetracycline formulation.

2. The method of claim 1, wherein the risk of cardiovascular disease is assessed by assessing a sample obtained from the patient for a marker of cardiovascular disease; obtaining an image of the patient; and/or subjecting the patient to a physical examination or test.

3. The method of claim 2, wherein the marker of cardiovascular disease is a marker of systemic inflammation.

4. The method of claim 3, wherein the marker of systemic inflammation is C-reactive protein (CRP), an interleukin-6 (IL-6) or another pro-inflammatory cytokine, or a gene whose expression is correlated with a marker of systemic inflammation.

5. The method of claim 4, wherein the marker of systemic inflammation is CRP and the gene whose expression is correlated therewith is a gene encoding hepatocyte nuclear factor-1α (HNF1A), a gene encoding a leptin receptor, or a gene encoding apolipoprotein E.

6. The method of claim 2, wherein the image is obtained by X-ray, nuclear imaging, or magnetic resonance imaging.

7. The method of claim 6, wherein the X-ray produces an image of the patient's chest, a chamber of the patient's heart, or the wall or lumen of a blood vessel within the patient.

8. The method of claim 2, wherein the physical examination comprises assessing the patient's blood pressure, cholesterol levels, body mass index, family history or electrical activity of the patient's heart and the physical test is a stress test.

9. The method of claim 1, wherein the tetracycline formulation is formulated for oral or intravenous administration.

10. A method of claim 1, wherein the tetracycline formulation comprises a non-antibacterial tetracycline.

11. The method of claim 10, wherein the non-antibacterial tetracycline is a chemically modified tetracycline compound or a pharmaceutically acceptable salt thereof.

12. The method of claim 11, wherein the chemically modified tetracycline compound differs from Formula I 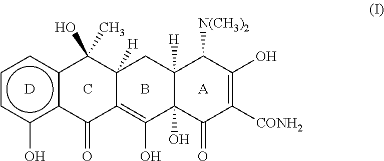
by a change to the basic ring system of or replacement of one or more of the substituents at positions 4, 10, 11, 12 or 12a according to Formula II:

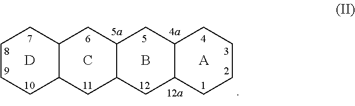

13. The method of claim 12, wherein the chemically modified tetracycline is
4-dedimethylaminotetracycline (CMT-1);
6-demethyl-6-deoxy-4-de(dimethylamino)tetracycline (CMT-3);
7-chloro-4-de(dimethylamino)tetracycline (CMT-4);
4-hydroxy-4-de(dimethylamino)-tetracycline (CMT-6);
4-de(dimethylamino)-12α-deoxytetracycline (CMT-7);
6-deoxy-5α-hydroxy-4-de(dimethylamino)tetracycline (CMT-8);
4-dedimethylamino-12α-deoxyanhydrotetracycline (CMT-9);
7-dimethylamino-6-demethyl-6-deoxy-4-de(dimethylamino)tetracycline (CMT-10);
4-dedimethylamino-5-oxytetracycline;
5α,6-anhydro-4-hydroxy-4-de(dimethylamino)tetracycline;
4-de(dimethylamino)-11-hydroxy-12α-deoxytetracycline;
12α-deoxy-4-deoxy-4-de(dimethylamino)tetracycline;
12α,4α-anhydro-4-de(dimethylamino)tetracycline;
6-α-benzylthiomethylenetetracycline;
6-fluoro-6-demethyltetracycline;
11α-chlorotetracycline; tetracyclinonitrile (CMT-2);
tetracycline pyrazole (CMT-5); or
CMT-308 (9-amino CMT-3).

14. The method of claim 1, wherein the tetracycline formulation comprises an antibacterial tetracycline or a pharmaceutically acceptable salt thereof at a sub-antibacterial concentration.

15. The method of claim 14, wherein the sub-antibacterial concentration is up to about 80% of the antibacterial amount of a tetracycline compound or a pharmaceutically acceptable salt thereof.

16. The method of claim 14, wherein the sub-antibacterial concentration is up to about 60% of an antibacterial amount of a tetracycline compound or a pharmaceutically acceptable salt thereof.

17. The method of claim 14, wherein the tetracycline compound is of the following Formula I: 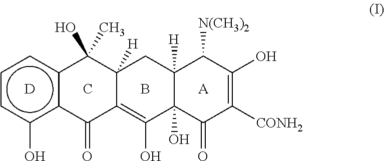
or is a pharmaceutically acceptable salt thereof.

18. The method of claim 14, wherein the tetracycline compound is an oxytetracycline or chlorotetracycline or a pharmaceutically acceptable salt thereof.

19. The method of claim 14, wherein the tetracycline compound is 7-dimethylaminotetracycline (minocycline) or 6α-deoxy-5-hydroxytetracycline (doxycycline) or a pharmaceutically acceptable salt thereof.

20. The method of claim 11, wherein the pharmaceutically acceptable salt is an acid addition salt.

21-32. (canceled)
